(19) United States
(12) Patent Application Publication
(10) Pub. No.: US 2020/0078210 A1
(43) Pub. Date: Mar. 12, 2020

(54) MULTIPLE USE ELECTRONIC HEAT THERAPY PATCHES

(71) Applicant: Multitech Medical Devices USA LLC, Holtsville, NY (US)

(72) Inventors: Shuh-Tsai Yang, New Taipei City (TW); John Weiss, Oakdale, NY (US)

(21) Appl. No.: 16/125,925

(22) Filed: Sep. 10, 2018

(51) Int. Cl. A61F 7/00

(52) U.S. Cl. CPC A61F 7/007; A61F 2007/0086; A61F 2007/0094; A61F 2007/0096; A61F 2007/0071

(57) ABSTRACT

A patch applied to the body to alleviate pain includes a heated section that is powered through a USB cable having temperature and time controls and includes a reusable adhesive for use multiple times.

### BACKGROUND

The use of heat to treat muscle and joint pain is well established. Heat therapy using heating pads increases blood circulation and elevates tension in joints and muscles to ease pain. Heating pads have been in existence for over 100 years. Common heat patches use a chemical exothermic reaction that is activated by air when removed from a sealed package. Other types include microwaveable gel packs placed in a holder. These devices enable heat therapy to be mobile instead of using a heating pad that is plugged into a stationary power source. Both the chemical type and the microwaveable type of thermal patches have a temperature change with time. For example, the chemical patch takes several minutes to activate and then for several hours increases in temperature and then for several hours decreases in temperature. The microwaveable type starts hot then decreases in temperature over time. Both the chemical type and the microwaveable type do not have accurate and consistent temperature control.

Another type of heat patch is an electronically-heated heat therapy patch. While the heat distribution with the electronic heat therapy patch does not have the same peaks and valleys as the chemical patches, it does offer a more consistent and even heat flow distribution which is better for the healing process. There are several heat patches using low voltage, as with a USB cable, that wrap around a body part using Velcro® to attach to the fabric or material of the wrap, such as the heat patches disclosed in US Patent Application Publication No. 2009/0127250 by Chang and US Patent Application Publication No. 2011/0065977 by Sham et al. Wraps are bulky, uncomfortable, and subject to shifting when a small area is to be treated. Using an adhesive has the advantage of keeping the patch in place with no accessories; however, adhesives are not reusable and limit the use to only one time.

### SUMMARY

In accordance with an aspect of the present disclosure, a multiple use electronically heated patch for application to a body is provided and includes a flexible outer layer, a flexible lower layer having a reusable adhesive bonded to a bottom surface of the lower layer for affixing to skin, a heater and control circuit disposed between the outer and lower layers, a USB power connector in electrical connection with the control circuit for supplying power, and an integrated circuit within the control circuit. The integrated circuit is configured to control the temperature of the heater and compute and control a time of use.

In aspects, the heater and reusable adhesive may overlap with one another.

In some aspects, the reusable adhesive may include a plurality of adhesive strips that are parallel and spaced-apart from one another.

In further aspects, the heater may include an array of insulative core members overlapping with the respective plurality of adhesive strips.

In other aspects, the heater may further include a resistive wire wrapped about the array of insulative core members.

In aspects, the patch may further include a heat-activated glue associated with the heater.

In some aspects, the reusable adhesive may be a silicone film.

In further aspects, the reusable adhesive may include a first section disposed on a first end of the bottom surface of the lower layer, and a second section disposed on a second end of the bottom surface of the lower layer, opposite the first end.

In another aspect of the present disclosure, a multiple use electronically heated patch for application to a body includes a flexible outer layer, a flexible lower layer coupled to the outer layer, at least one adhesive strip bonded to a bottom surface of the lower layer for affixing to skin, and a heater disposed between the outer and lower layers and overlapping with the at least one adhesive strip.

In aspects, the at least one adhesive strip may be a plurality of adhesive strips that are parallel and spaced-apart from one another.

In other aspects, the patch may further include a control circuit disposed between the outer and lower layers, a USB power connector in electrical connection with the control circuit for supplying power, and an integrated circuit within the control circuit and configured to control the temperature of the heater and compute and control a time of use.

Further details and aspects of exemplary embodiments of the present disclosure are described in more detail below with reference to the appended figures.

As used herein, the terms parallel and perpendicular are understood to include relative configurations that are substantially parallel and substantially perpendicular up to about + or −10 degrees from true parallel and true perpendicular.

### BRIEF DESCRIPTION OF THE DRAWINGS

Embodiments of the present disclosure are described herein with reference to the accompanying drawings, wherein:

FIG. 1A is a top view of an exemplary embodiment of an electric heat patch for applying heat therapy to a user;

FIG. 1B is a bottom view of the patch of FIG. 1A;

FIG. 1C is an exploded view of the patch of FIG. 1A, illustrating the internal construction thereof;

FIG. 2A is a bottom view of an alternate embodiment of an electric heat patch;

FIG. 2B is a top view, with an upper fabric layer removed, of the patch of FIG. 2A;

FIG. 2C is top view of the patch of FIG. 2A;

FIG. 3 is a control circuit diagram;

FIG. 4 is a flow chart of the control program; and

FIG. 5 is a time temperature chart comparing the traditional chemical heat patches with the electronic thermal patch of the present disclosure.

### DETAILED DESCRIPTION

Embodiments of the presently disclosed electric heat patches are described in detail with reference to the drawings, in which like reference numerals designate identical or corresponding elements in each of the several views.

Modern heating pads employ safety circuits to protect the user as they are powered by 120 or 240 volts AC that poses a risk to the user. Often the availability of AC line power is not present, for these cases a mobile type of heat is applied, usually in the form of a chemical patch that may last up to eight hours. In today's modern environment, low voltage power is commonly available through a USB port found in laptop and personal computers, power banks used for extended charging of phones and tablets, car dash, adapters to cigarette lighter receptacles and AC adapters.

The present disclosure provides embodiments of a self-stick electronic heat patch for multiple use. The heat patches incorporate a microprocessor to control the temperature and time of use. The electronics have the benefit of being able to monitor the safety of the heating pad by checking the integrity of the heater and the power switching circuit. The patch has a USB connector to connect to a computer, power adaptor, or power bank for mobile use. The heating element is held aligned with the body part to be treated using a medical grade silicon film that may not have an added glue or adhesive. The multiple use aspect of the present disclosure reduces the cost of treatment as compared to chemical patches as well as offering tremendous environmental friendly dynamics to this system. Without chemicals to be concerned about, the electronic multiple use patch of the present disclosure is both environmentally safe and nontoxic.

The electronic heating patches of the present disclosure adhere to any body part to bring pain relief and comfort to the user greatly eliminating any chance of shock or bodily harm. The electronic patch, sometimes referred to herein as an e-Patch, is described below and is capable of adhering to the skin without adhesives that wear out or leave a residue of glue on the skin. Two embodiments are disclosed to describe the present invention, the shape and size are shown for illustrative purpose and the functionality is not limited by the particular configurations of the embodiments.

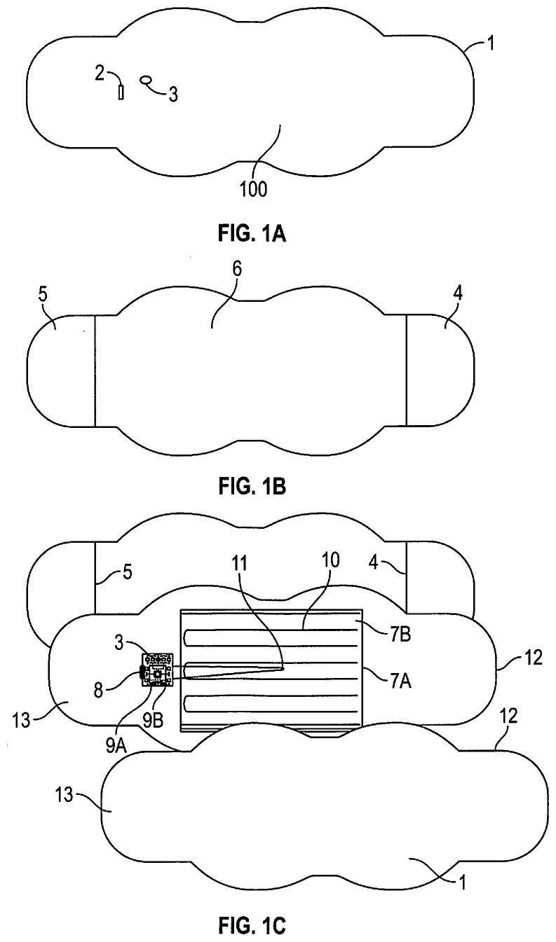

With reference to FIGS. 1A-1C, an exemplary embodiment of a multi-use, electronic heat patch (“e-Patch”) is illustrated and is generally designated 100. The top view shown in FIG. 1A of the e-Patch 100 is shown having the open end of a USB connector 2, such as, for example, a micro USB connector, a mini USB connector, or a standard USB connector, and a power indicator LED 3 that is on when the e-Patch 100 is in the heating mode. The LED 3 is actually mounted to a printed circuit board 9A (described later), and the LED shines through the outer fabric of the e-Patch 100. This LED 3 also indicates if the e-Patch 100 is in safe mode by blinking in a predetermined sequence.

The underside of the e-Patch 100, FIG. 1B, has first and second sections of reusable adhesive 4 and 5 for attaching to the skin. The reusable adhesive 4, 5 may be a silicone film specially designed to attach to the skin without the use of an adhesive. In some aspects, the reusable adhesive may be a silicone film of the type sold under the name “3M™ Kind Removal Silicone Tape” by 3M company corp. The silicone film 4 and 5 is of medical grade and tested for compatibility and long term toxicity. Other uses for this medical grade silicone include artificial skin used in a similar manner as a band aid or artificial scab. The e-Patch 100 is applied by stretching over the area to be treated and pressing the reusable adhesive sections 4, 5 to make an intimate attachment to the skin. The e-Patch 100 may be attached to uneven surfaces and has little effect by hair that may be on the skin. When the e-Patch 100 is removed, no residue is left behind and neither hair nor an open wound will be affected by the removal.

The reusable adhesive 4, 5 is bonded to a bottom surface of a flexible bottom or lower layer 6 of the e-Patch 100. The e-Patch 100 further includes an upper or outer layer 1 bonded or otherwise coupled to the lower layer 6. The upper and lower layers 1, 6 of the e-Patch 1 are preferably made of a soft polyester fabric. Other suitable types of fabrics are also contemplated.

The internal components of the e-Patch 100 are illustrated in FIG. 1C. A heater 10 is attached to the upper fabric 1 and lower fabric 6 by a heat-activated glue (not explicitly shown) having similar properties to hot melt adhesive glue. The heater 10 includes a heater wire alloy 7A fixed in a fabric matrix in a serpentine pattern equally spaced to provide uniform heat to the treat area. The heater wire 7A may be helically wrapped around a thin core member 7B (e.g., plastic, rubber, or any other suitable insulative material) and woven through the matrix of the internal heater 10. The thin core member 7B may be an array of insulative core members that are spaced-apart and parallel with one another. In some aspects, the heater wire 7A may be a resistive wire heating element. In other aspects, the heater wire 7A may have positive temperature of resistance characteristics, whereby the resistance increases with increasing temperature and may functionally double as a temperature sensor. In aspects, the heater wire 7A may assume any suitable pattern. The heater wire 7 and a power USB connector 8 are electrically attached to a printed circuit board 9A.

The circuit board 9A has a control circuit, such as, for example, an integrated circuit 9B (e.g., a microprocessor uP), and a temperature sensor 11 electrically connected to the printed circuit board 9A. The printed circuit board 9A may be made of a rigid fiberglass type or as a flexible type as is known by the industry. As described above, a surface mount LED 3 is soldered to the printed circuit board 9A. The heater assembly 10 is sandwiched between the upper and lower layers 1, 6 of fabric and bonded by adhesive. The heat-activated glue may be used for this purpose and activates the adhesive within the heater 10 to form a bonded envelope. The heat-activated glue in the heater 10 also improves the heat transfer to the surfaces of the e-Patch 100 envelope. The silicone adhesive film 4 and 5 is then attached to the lower fabric forming two tabs 12 and 13 for attachment to the user in this first embodiment.

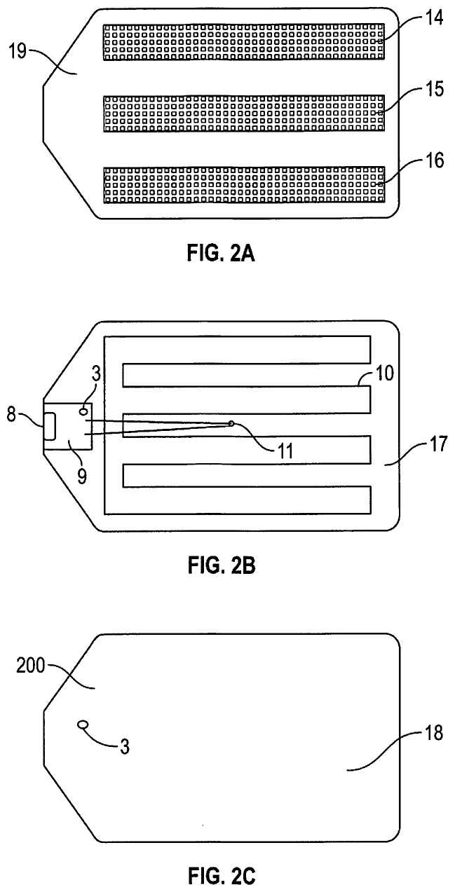

With reference to FIGS. 2A, 2B, and 2C, another embodiment of a multiple use electronic patch 200 is illustrated, similar to the e-Patch 100 described above. Due to the similarities between the e-patch 200 of the present embodiment and the e-Patch 100 described above, only those elements of the e-patch 200 deemed necessary to elucidate the differences from e-Patch 100 described above will be described in detail.

The e-patch 200 includes a plurality of silicone adhesive strips 14, 15 and 16 bonded to a lower layer 17 of the e-patch 200 in the same manner as the first embodiment. The internal construction has the same combination of heater assembly 10 with heater element 7, temperature sensor 11, and printed circuit board 9A with LED 3. The heater assembly 10 may be heat bonded to the lower layer 17 (FIG. 2B) and a top layer 18 (FIG. 2C). The portion 19 of the e-Patch 200 that does not have the adhesive 14, 15, 16 is used to attach the USB cable (not shown) and also to remove the e-patch 200 from the skin. The adhesive strips 14, 15 and 16 are coincident (e.g., overlapping) with the internal heater assembly 10 and serves at least two purposes. The adhesive strips 14, 15, 16 provide a direct heat transfer path from the heating assembly 10 to the skin and also prevents the e-patch 200 from lifting off the skin surface when used on a joint that can change shape such as the inside of the knee, inside of the elbow, wrist, neck ankle, hip or any part of the body that flexes.

The e-patch 200 can be worn on any surface of the body, when used in a location that is hard to reach, such as the upper or middle of a user's back, the USB cable may remain attached and routed through clothes to a convenient opening ready to plug in to a USB power source. This use method is convenient as the e-patch 200 only needs to be removed when exposed to the elements such as in the shower or bath, swimming, hot tub or activity that may cause the e-patch 200 to become dislodged. When used with a USB power bank the combination allows for totally mobile use. Since the e-patch 200 is designed to attach flat to the body it is not subject to bunching or folding, no hot spots occur due to the heating element is not able to fold over upon itself. The e-patch 200 is controlled for the best therapeutic temperature.

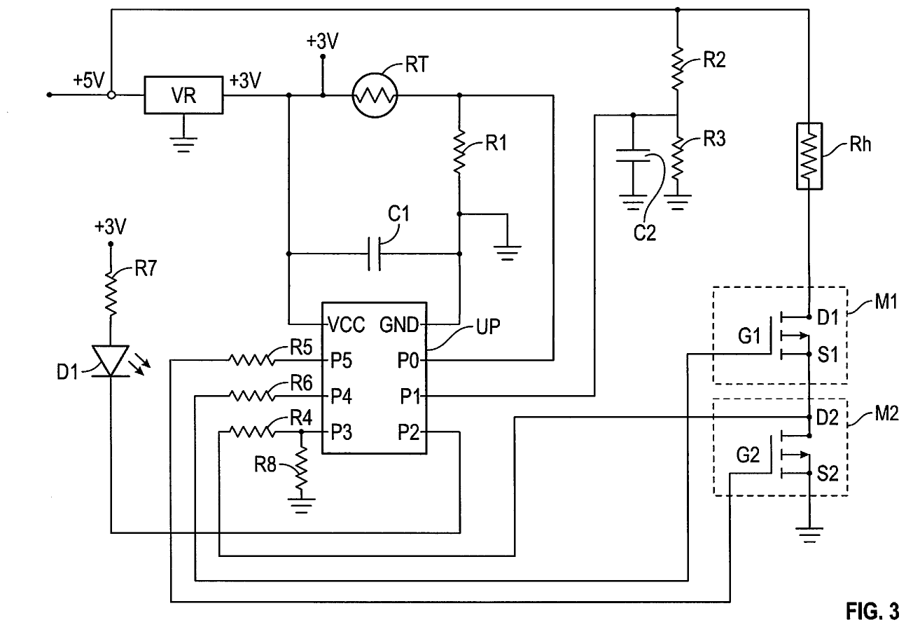

The printed circuit board 9A is populated with a microprocessor uP (FIG. 3) and dual inline MOSFET switch. Referring to FIG. 3, the microprocessor uP is powered from a 3 volt voltage regulator VR providing stable power voltage for the microprocessor uP and the temperature sensor Rt. A power filter capacitor C1 reduces the noise induced by the microcontroller uP onto the thermistor circuit.

The sensor is a low cost thermistor that forms a voltage divider with resistor R1. The output voltage of the voltage divider is read by the analog port P0 of the microprocessor uP. The input port P0 is configured as an input of an analog to digital converter, where the voltage at P0 is translated to a digital equivalent of the patch temperature.

The heater wire Rh is powered through the action of a pair of series connected MOSFETs M1 and M2. With continued reference to FIG. 3, the power delivered to the heater is controlled as follows. Output port P5 switches high providing a high potential to the gate G2 through a resistor R5 of MOSFET M2 causing conduction between the source terminal S2 and the drain terminal D2, simultaneously the output port P4 provides current to the gate G1 through the resistor R6 causing conduction of MOSFET M1 between S1 and D1. The drain of the MOSFET M2 at D2 is connected in series to the source of the MOSFET M1 S1 switching the heater Rh to ground. Safety is achieved by driving each MOSFET switch separately and in the off state of each MOSFET checking the status of the output voltage through resistor R4 into the analog to digital port P3. If either MOSFET M1 or MOSFET M2 is shorted the microcontroller will prevent the other MOSFET switch from conducting. The power voltage is monitored through the voltage divider R2 and R3 with C2 providing signal stability, if the input power voltage drops below a preset value, e.g. 3.5 volts, the microprocessor uP will prevent the switching action of the MOSFETs.

The microprocessor uP continuously monitors the condition of the power switch and the temperature sensor Rt, in case of a failure the uP prevents heating the heater Rt by setting both gates G1 and G2 of the dual MOSFET low, and signals the user by blinking the LED D1. A current limiting resistor R7 is in series with LED D1 to control the current through the LED D1. When the circuit is in the heating mode the LED D1 is powered continuously.

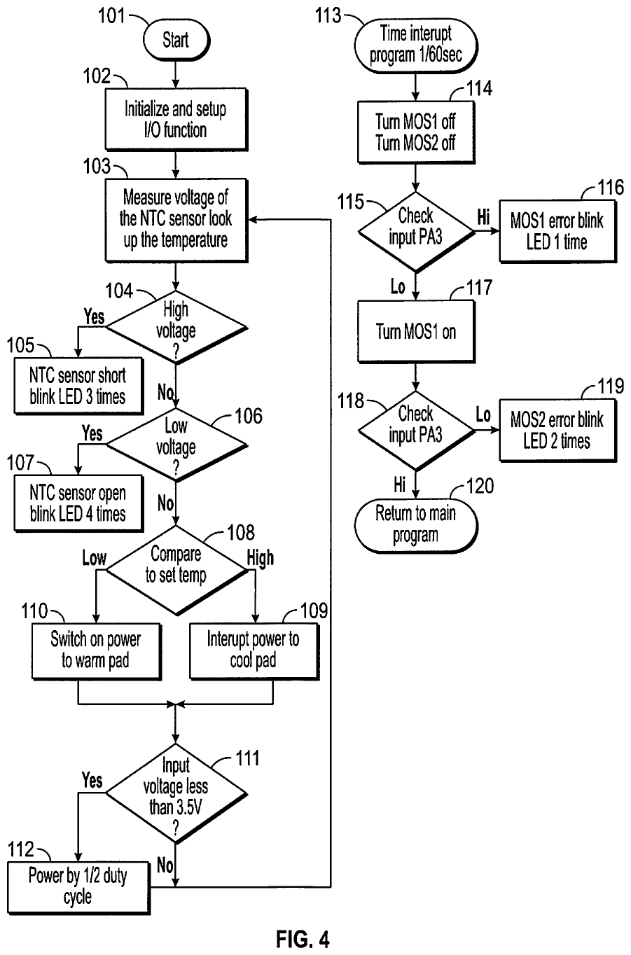

The microprocessor uP calculates the temperature for temperature control, checks the integrity of the temperature sensor Rt, checks the integrity of the dual MOSFET switch, FIG. 3 shown as MOS1 and MOS2, and checks the integrity of the heater Rh. The flow chart, FIG. 4, describes these functions.

With reference to FIG. 4, upon startup 101 the program initializes and resets input and output functions 102. The voltage of the temperature sensor Rt at the junction of the resistor divider Rt-R1 is input on port P1, the microprocessor uP performs an analog to digital conversion and compares to a lookup table 103. If the voltage is over a maximum threshold voltage 104 then the sensor is at least partially shorted and an error is detected resulting in blinking the LED 3 times 105. If the voltage is not over the maximum threshold then the voltage is checked for low voltage threshold 106, if below then the sensor is open and an error is detected resulting in blinking the LED 4 times 107. If the voltage is between the maximum threshold and the minimum threshold then the value in the lookup table is compared to the predetermined set temperature 108. Greater than the set temperature interrupts the power to the e-Patch heater 109 if the voltage is not greater than the set temperature then switch power on, MOS1 and MOS2 on, to the e-Patch Heater 110. The 5 volt input voltage is then compared to 3.5 volts 111, if less than 3.5V then the e-Patch is powered by ½ duty cycle 112 to extend the life of the power source. The routine is returned to block 103 forming a temperature control loop. A separate routine is shown in FIG. 4 for the safety interrupt. Periodically an interrupt routine is called to test the integrity of the two MOSFET switches. Here 113 sets the time interrupt at 1/60th of a second 113 and turns both MOS1 and MOS2 off 114. The voltage at the junction of the series connection of MOS1 and MOS2 is checked at the input PA3 115, if the voltage is high then the switch MOS1 is considered to be short circuited and the LED blinks 3 times continuously 116 and the routine is not returned to the main program. If the voltage on PA3 is low the MOS1 switch is turned on 117 and PA3 is checked again 118. This time if PA3 is low then a short of MOS2 is determined and the LED blinks 4 times continuously 119 not returning to the main program. If the second check of PA3 is high then both MOS1 and MOS2 are not shorted and no risk of unsafe condition exists and the interrupt is ended returning to the main program 120.

The description of the circuit and program logic is presented as an example of the control and safety aspects of the present invention. Other methods of temperature and circuit integrity are conceivable such as using a heater element having a Positive Temperature coefficient as both the heater and sensor and checking the integrity of the heater then becomes apparent. Another safety feature employs a use timer so that the use time for a single thermal treatment is limited by way of an auto off timer and also the total accumulated use time can be used to limit the age of the system due to the wear upon the construction of both the patch and the multiple connections made to the USB connector.

A microprocessor with communication capability can be combined with a Bluetooth or WiFi device to remotely communicate with a smart phone or computer or the communication ability of the USB connection can alternatively be used with a computer to enable another device to access parameters in the program. This can be used to adjust the set temperature and the auto off time to customize the heat therapy.

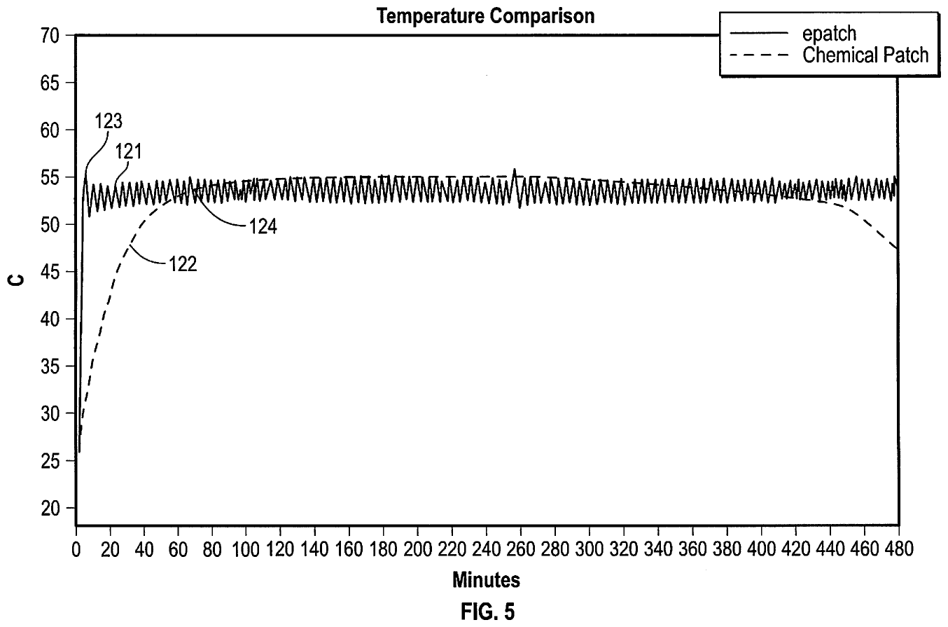

FIG. 5 shows a time temperature comparison between the e-Patch 100 or 200 of the present invention and a popular chemical patch. The on off temperature response of the e-Patch 100 or 200 is enhanced for illustration, the on off differential is a function of the hysteresis in the temperature control cycle. The e-Patch's 100 or 200 temperature profile is shown as the plot 121 and the temperature profile of the chemical patch is shown as the plot 122. Note the e-Patch's 100 or 200 temperature arrives at the set temperature in only 4 minutes 123 and the chemical patch arrives at the maximum temperature in over 60 minutes. The advantage of rapidly obtaining the desired temperature is obvious, especially if the heat therapy time is only one or two hours. The chemical patch also has a fixed use time that may be longer than the optimal treatment time, only the removal and disposal of the chemical patch can shorten the heat treatment period, therefore the e-Patch 100 or 200 is much more cost-efficient.

The advantages of the e-Patches 100 or 200 of the present disclosure over traditional chemical patches and electric heat patches are obvious in light of the illustrations and description provided herein. The scope of this invention is not limited by the embodiments described either by shape, size or function.

It will be understood that various modifications may be made to the embodiments disclosed herein. Therefore, the above description should not be construed as limiting, but merely as exemplifications of various embodiments. Those skilled in the art will envision other modifications within the scope and spirit of the claims appended thereto.

1. A multiple use electronically heated patch for application to a body, comprising:
a flexible outer layer;
a flexible lower layer having a reusable adhesive bonded to a bottom surface of the lower layer for affixing to skin;
a heater and control circuit disposed between the outer and lower layers;
a USB power connector in electrical connection with the control circuit for supplying power; and
an integrated circuit within the control circuit, wherein the integrated circuit is configured to control the temperature of the heater and compute and control a time of use.

2. The multiple use electronically heated patch according to claim 1, wherein the heater and reusable adhesive overlap with one another.

3. The multiple use electronically heated patch according to claim 1, wherein the reusable adhesive includes a plurality of adhesive strips that are parallel and spaced-apart from one another.

4. The multiple use electronically heated patch according to claim 3, wherein the heater includes an array of insulative core members overlapping with the respective plurality of adhesive strips.

5. The multiple use electronically heated patch according to claim 4, wherein the heater further includes a resistive wire wrapped about the array of insulative core members.

6. The multiple use electronically heated patch according to claim 1, further comprising heat-activated glue associated with the heater.

7. The multiple use electronically heated patch according to claim 1, wherein the reusable adhesive is a silicone film.

8. The multiple use electronically heated patch according to claim 1, wherein the reusable adhesive includes:
a first section disposed on a first end of the bottom surface of the lower layer; and
a second section disposed on a second end of the bottom surface of the lower layer, opposite the first end.

9. A multiple use electronically heated patch for application to a body, comprising:
a flexible outer layer;
a flexible lower layer coupled to the outer layer;
at least one adhesive strip bonded to a bottom surface of the lower layer for affixing to skin;
a heater disposed between the outer and lower layers and overlapping with the at least one adhesive strip; and
a control circuit disposed between the outer and lower layers.

10. The multiple use electronically heated patch according to claim 9, wherein the at least one adhesive strip is a plurality of adhesive strips that are parallel and spaced-apart from one another.

11. The multiple use electronically heated patch according to claim 10, wherein the heater includes an array of insulative core members overlapping with the respective plurality of adhesive strips.

12. The multiple use electronically heated patch according to claim 11, wherein the heater further includes a resistive wire wrapped about the array of insulative core members.

13. The multiple use electronically heated patch according to claim 9, further comprising:
a USB power connector in electrical connection with the control circuit for supplying power; and
an integrated circuit within the control circuit and configured to control a temperature of the heater and compute and control a time of use.

14. The multiple use electronically heated patch according to claim 9, further comprising heat-activated glue associated with the heater.

15. The multiple use electronically heated patch according to claim 9, wherein the at least one adhesive strip is a silicone film.

16. The multiple use electronically heated patch according to claim 9, wherein the control circuit includes a Bluetooth module for remotely adjusting a temperature setting of the patch.
